(19) United States
(12) Patent Application Publication
(10) Pub. No.: US 2019/0005640 A1
(43) Pub. Date: Jan. 3, 2019

(54) PHYSIOLOGY MAPS FROM MULTI-PARAMETRIC RADIOLOGY DATA

(71) Applicant: General Electric Company, Schenectady, NY (US)

(72) Inventors: Dattesh Dayanand Shanbhag, Bangalore (IN); Mirabela Rusu, Ballston Spa, NY (US); Sandeep Narendra Gupta, Niskayuna, NY (US)

(21) Appl. No.: 15/640,909

(22) Filed: Jul. 3, 2017

(51) Int. Cl. G06T 7/00; G01R 33/48; A61B 5/055; A61B 6/03; A61B 8/08; A61B 6/00; A61B 8/00; A61B 5/00

(52) U.S. Cl. CPC G06T 7/0012; G06T 2207/10104; A61B 5/055; A61B 6/032; A61B 6/037; A61B 8/5246; A61B 6/5235; A61B 8/463; A61B 6/463; A61B 5/743; A61B 5/4878; G06T 2207/10088; G06T 2207/10132; G06T 2207/10081; G01R 33/4808

(57) ABSTRACT

The disclosed approach employs a generic methodology for transforming individual modality specific multi-parametric data into data, e.g., maps or images, which provides direct insight into the underlying physiology of the tissue. This may facilitate better clinical evaluation of the disease data as well as help non-imaging technologists and scientist to directly correlate imaging findings with basic biological phenomenon being studied with imaging.

### TECHNICAL AREA

The subject matter disclosed herein relates to interpretation of imaging data.

### BACKGROUND

Non-invasive imaging technologies allow images of the internal structures or features of a patient or subject to be obtained. In particular, such non-invasive imaging technologies rely on various physical principles, such as the paramagnetic properties of tissues within the subject, the differential transmission of X-ray photons through an imaged volume, the emission of gamma rays by a radiopharmaceutical differentially distributed in the body, or the reflection of acoustic waves by structures within the body, to acquire data and to construct images or otherwise represent the internal features of the subject.

In clinical practice, clinicians and biologists are primarily interested in interpreting or deducing the physiological or biological interpretation of such imaging data. Viewing and interpretation of the native imaging data, however, may be limited by the inherent contrast mechanisms of the imaging modality (e.g. Hounsfield units from CT, T1W contrast or T2W contrast form MRI or SUV image from PET). Consequently, to properly analyze image data a reviewer needs to understand the physiological underpinnings of each of the imaging inherent contrast mechanisms, map each voxel or region of interest in each of the images to spatially overlapping physiological components and then make a clinical decision or other relevant analytic outcome. Such assessments may be particularly difficult for reviewers not trained in interpreting such image data.

### BRIEF DESCRIPTION

The present approach employs a generic methodology for transforming individual modality specific multi-parametric data into data, e.g., maps or images, which provides direct insight into the underlying physiology of the tissue. This may facilitate better clinical evaluation of the disease data as well as help non-imaging technologists and scientist to directly correlate imaging findings with basic biological phenomenon being studied with imaging. For example, untrained reviewers, may be confused by numerous contrast mechanisms of imaging data, their values, and their interpretation when studying biological processes (e.g., proliferation in tumors or gene expression involved in inflammation). So, presenting the imaging data in a format which can be directly correlated to biology (e.g., necrosis, edema, and so forth) will accelerate research activities using different radiological imaging modalities and wider acceptance in the community.

Certain embodiments commensurate in scope with the originally claimed subject matter are summarized below. These embodiments are not intended to limit the scope of the claimed subject matter, but rather these embodiments are intended only to provide a brief summary of possible embodiments. Indeed, the invention may encompass a variety of forms that may be similar to or different from the embodiments set forth below.

In one aspect, a method is provided for generating a physiology labeled image. In accordance with this aspect, the method includes the step of acquiring two or more multi-parametric images of a subject. The two or more multi-parametric images are acquired using different imaging protocols. A data reduction analysis is performed on the two or more multi-parametric images. The outputs of the data reduction analysis comprises computational products of the two or more images into one or more physiological components. The physiology labeled image is generated based on the computational products. The physiology labeled image is displayed for review.

In accordance with a further aspect, an image processing system is provided. The image processing system includes a processor configured to execute executable instructions and a memory configured to store executable instructions that, when executed by the processor, cause act to be performed comprising: acquiring or accessing two or more multi-parametric images of a subject, wherein the two or more multi-parametric images are acquired using different imaging protocols; performing a data reduction analysis on the two or more multi-parametric images, wherein the outputs of the data reduction analysis comprises computational products of the two or more images into one or more physiological components; generating a physiology labeled image based on the computational products; and displaying the physiology labeled image for review

In accordance with an additional aspect, one or more non-transitory computer readable media are provided. The media encode routines which, when executed, cause acts to be performed comprising: acquiring two or more multi-parametric images of a subject, wherein the two or more multi-parametric images are acquired using different imaging protocols; performing a data reduction analysis on the two or more multi-parametric images, wherein the outputs of the data reduction analysis comprises computational products of the two or more images into one or more physiological components; generating a physiology labeled image based on the computational products; and displaying the physiology labeled image for review

### BRIEF DESCRIPTION OF THE DRAWINGS

These and other features, aspects, and advantages of the present invention will become better understood when the following detailed description is read with reference to the accompanying drawings in which like characters represent like parts throughout the drawings, wherein:

FIG. 1 illustrates an embodiment of a magnetic resonance imaging (MRI) system, in accordance with an aspect of the present disclosure;

FIG. 2 depicts multi-parametric MRI oncology data mapped to physiology components, in accordance with an aspect of the present disclosure;

FIG. 3 depicts multi-parametric MRI oncology data factorized to two physiology components, in accordance with an aspect of the present disclosure; and

FIG. 4 depicts basis vectors from a non-negative matrix factorization study, in accordance with an aspect of the present disclosure.

### DETAILED DESCRIPTION

One or more specific embodiments will be described below. In an effort to provide a concise description of these embodiments, all features of an actual implementation may not be described in the specification. It should be appreciated that in the development of any such actual implementation, as in any engineering or design project, numerous implementation-specific decisions must be made to achieve the developers' specific goals, such as compliance with system-related and business-related constraints, which may vary from one implementation to another. Moreover, it should be appreciated that such a development effort might be complex and time consuming, but would nevertheless be a routine undertaking of design, fabrication, and manufacture for those of ordinary skill having the benefit of this disclosure.

When introducing elements of various embodiments of the present embodiments, the articles “a,” “an,” “the,” and “said” are intended to mean that there are one or more of the elements. The terms “comprising,” “including,” and “having” are intended to be inclusive and mean that there may be additional elements other than the listed elements. Furthermore, any numerical examples in the following discussion are intended to be non-limiting, and thus additional numerical values, ranges, and percentages are within the scope of the disclosed embodiments.

In accordance with the present approach, a methodology for generating and labelling biologically or physiologically relevant data from provided multi-parametric data is provided. The labeled biologically or physiologically relevant data may then be displayed to facilitate clinical interpretation or other analysis, such as providing the data in a suitable format for further analysis.

In one implementation, this process is accomplished by using various data factorization methods such as non-negative matrix factorization (NNMF), convex analysis of mixtures with non-negative sources (CAMNS), and so forth, for data separation. The output of factorization methods and their signatures are then used to label regions of the image data as belonging to one or more physiological states, processes, or structures of interest.

To facilitate explanation of the present approach, an example is described herein related to the use of multi-parametric data corresponding to what might be obtained as part of a magnetic resonance imaging (MRI) oncology protocol. As used herein, the term “multi-parametric” relates to a plurality of images that can be collected, such as for a patient examination. In this context, each image (for example, diffusion images, contrast-enhanced T1 weighted images, and so forth) constitutes different “channels” or information. Though MRI examples are discussed herein, it is to be understood that the present approach may be similarly implemented using different types of data datasets, including datasets acquired using other MRI protocols and/or other imaging modality types and protocols, including computed tomography (CT), tomosynthesis, mammography, ultrasound, positron emission tomography (PET), single photon emission computed tomography (SPECT), and so forth. Thus, the present approach is not restricted to MRI or tumor assessment, but to more generic physiological representations of multi-parametric (mp)-MRI data as well as other protocols and modality types. Therefore, even in the MRI context, the present approach can be used across disease conditions since physiology-MRI relationships are maintained across disease conditions. However, as may be appreciated, the manifestations in individual MRI images change across disease conditions, however, such variance in manifestations will be understood in the context of a given protocol by those skilled in the art (e.g., in tumor, restricted diffusion is related to aggressiveness of tumor, while in stroke it is related to regions of infarction; but physiologically it is still represented as increased cellularity).

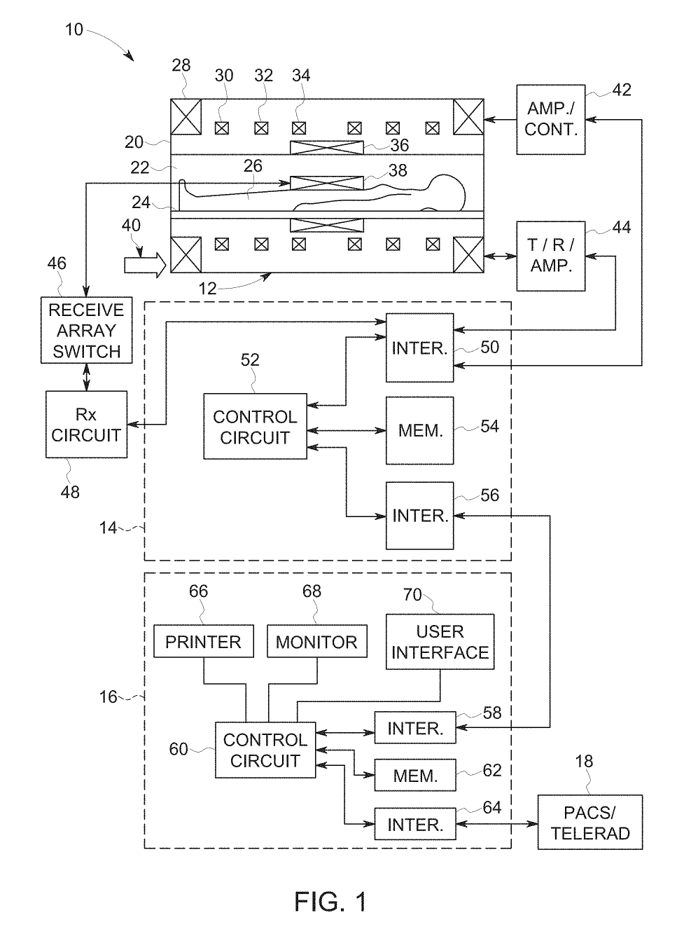

The examples described herein may be performed by a magnetic resonance imaging (MRI) system in which specific imaging routines (e.g., diffusion MRI sequences) are initiated by a user (e.g., a radiologist). Thus, the MRI system may perform data acquisition, data construction, and in certain instances, image synthesis in accordance with the techniques discussed herein. Accordingly, to provide context with respect to the present MRI examples, an MRI system 10 is described in FIG. 1. A magnetic resonance imaging system 10 is illustrated schematically as including a scanner 12, scanner control circuitry 14, and system control circuitry 16. According to the embodiments described herein, the MRI system 10 is generally configured to perform MR imaging.

System 10 additionally includes remote access and storage systems or devices such as picture archiving and communication systems (PACS) 18, or other devices such as teleradiology equipment so that data acquired by the system 10 may be accessed on- or off-site. In this way, MR data may be acquired, followed by on- or off-site processing and evaluation. While the MRI system 10 may include any suitable scanner or detector, in the illustrated embodiment, the system 10 includes a full body scanner 12 having a housing 20 through which a bore 22 is formed. A table 24 is moveable into the bore 22 to permit a patient 26 to be positioned therein for imaging selected anatomy within the patient.

Scanner 12 includes a series of associated coils for producing controlled magnetic fields for exciting the gyromagnetic material within the anatomy of the subject being imaged. Specifically, a primary magnet coil 28 is provided for generating a primary magnetic field, B0, which is generally aligned with the bore 22. A series of gradient coils 30, 32, and 34 permit controlled magnetic gradient fields to be generated for positional encoding of certain of the gyromagnetic nuclei within the patient 26 during examination sequences. A radio frequency (RF) coil 36 is configured to generate radio frequency pulses for exciting the certain gyromagnetic nuclei within the patient. In addition to the coils that may be local to the scanner 12, the system 10 also includes a set of receiving coils 38 (e.g., an array of coils) configured for placement proximal (e.g., against) to the patient 26. As an example, the receiving coils 38 can include cervical/thoracic/lumbar (CTL) coils, head coils, single-sided spine coils, and so forth. Generally, the receiving coils 38 are placed close to or on top of the patient 26 so as to receive the weak RF signals (weak relative to the transmitted pulses generated by the scanner coils) that are generated by certain of the gyromagnetic nuclei within the patient 26 as they return to their relaxed state.

The various coils of system 10 are controlled by external circuitry to generate the desired field and pulses, and to read emissions from the gyromagnetic material in a controlled manner. In the illustrated embodiment, a main power supply 40 provides power to the primary field coil 28. A driver circuit 42 provides power to pulse the gradient field coils 30, 32, and 34. Such a circuit may include amplification and control circuitry for supplying current to the coils as defined by digitized pulse sequences output by the scanner control circuit 14, which in one embodiment may be a diffusion imaging module. Another control circuit 44 is provided for regulating operation of the RF coil 36. Circuit 44 includes a switching device for alternating between the active and inactive modes of operation, wherein the RF coil 36 transmits and does not transmit signals, respectively. Circuit 44 also includes amplification circuitry configured to generate the RF pulses. Similarly, the receiving coils 38 are connected to switch 46, which is capable of switching the receiving coils 38 between receiving and non-receiving modes. Thus, the receiving coils 38 resonate with the RF signals produced by relaxing gyromagnetic nuclei from within the patient 26 while in the receiving mode, and they do not resonate with RF energy from the transmitting coils (i.e., coil 36) so as to prevent undesirable operation while in the non-receiving mode. Additionally, a receiving circuit 48 is configured to receive the data detected by the receiving coils 38, and may include one or more multiplexing and/or amplification circuits.

It should be noted that while the scanner 12 and the control/amplification circuitry described above are illustrated as being coupled by a single line, that many such lines may occur in an actual instantiation. For example, separate lines may be used for control, data communication, and so on. Further, suitable hardware may be disposed along each type of line for the proper handling of the data. Indeed, various filters, digitizers, and processors may be disposed between the scanner and either or both of the scanner and system control circuitry 14, 16. By way of non-limiting example, certain of the control and analysis circuitry described in detail below, although illustrated as a single unit, includes additional hardware such as image reconstruction hardware configured to perform the data processing techniques described herein.

As illustrated, scanner control circuit 14 includes an interface circuit 50, which outputs signals for driving the gradient field coils and the RF coil and for receiving the data representative of the magnetic resonance signals produced in examination sequences. The interface circuit 50 is coupled to a control and analysis circuit 52. The control and analysis circuit 52 executes the commands for driving the circuit 42 and circuit 44 based on defined protocols selected via system control circuit 16. Control and analysis circuit 52 also serves to receive the magnetic resonance signals and performs subsequent processing before transmitting the data to system control circuit 16. Scanner control circuit 14 also includes one or more memory circuits 54, which store configuration parameters, pulse sequence descriptions, examination results, and so forth, during operation.

Interface circuit 56 is coupled to the control and analysis circuit 52 for exchanging data between scanner control circuit 14 and system control circuit 16. In certain embodiments, the control and analysis circuit 52, while illustrated as a single unit, may include one or more hardware devices. The system control circuit 16 includes an interface circuit 58, which receives data from the scanner control circuit 14 and transmits data and commands back to the scanner control circuit 14. The interface circuit 58 is coupled to a control and analysis circuit 60 which may include a CPU or other microprocessor architecture that may be present in a multi-purpose or application specific computer or workstation. Control and analysis circuit 60 is coupled to a memory circuit 62 to store programming code for operation of the MRI system 10 and to store the processed image data for later reconstruction, display and transmission. The programming code may execute one or more algorithms that, when executed by a processor, are configured to perform reconstruction of acquired data and may further include algorithms for generating images in accordance with the techniques discussed herein.

An additional interface circuit 64 may be provided for exchanging image data, configuration parameters, and so forth with external system components such as remote access and storage devices 18. Finally, the system control and analysis circuit 60 may include various peripheral devices for facilitating operator interface and for producing hard copies of the reconstructed images. In the illustrated embodiment, these peripherals include a printer 66, a monitor 68, and user interface 70 including devices such as a keyboard or a mouse.

It should be noted that the MRI system described is provided merely as an example, and other system types, such as so-called “open” MRI systems may also be used. Similarly, such systems may be rated by the strength of their primary magnet, and any suitably rated system capable of carrying out the data acquisition and processing described below may be employed. Indeed, at least a portion of the methods disclosed herein may be performed by the system 10 described above with respect to FIG. 1. That is, the MRI system 10 may perform the acquisition and reconstruction techniques described herein. It should be noted that subsequent to the acquisition of data, the system 10 may simply store the acquired data for later access locally and/or remotely, for example in a memory circuit (e.g., memory 62). Thus, when accessed locally and/or remotely, the acquired data may be manipulated by one or more processors contained within an application-specific or general-purpose computer. The one or more processors may access the acquired data and execute routines stored on one or more non-transitory, machine readable media collectively storing instructions for performing methods including the multi-shot, multi-acquisition image averaging techniques described herein. As an example, the methods described herein may be performed by control and analysis circuitry associated with or otherwise communicatively coupled to the MR scanner 12.

With the preceding in mind, the present approach generates and labels biologically or physiologically relevant data from input multi-parametric data, such as may be generated using an imaging system such as that shown in FIG. 1. The labeled biologically or physiologically relevant data constitutes a physiologically meaningful image that may then be displayed to facilitate clinical interpretation or other analysis, such as providing the data in a suitable format for further analysis

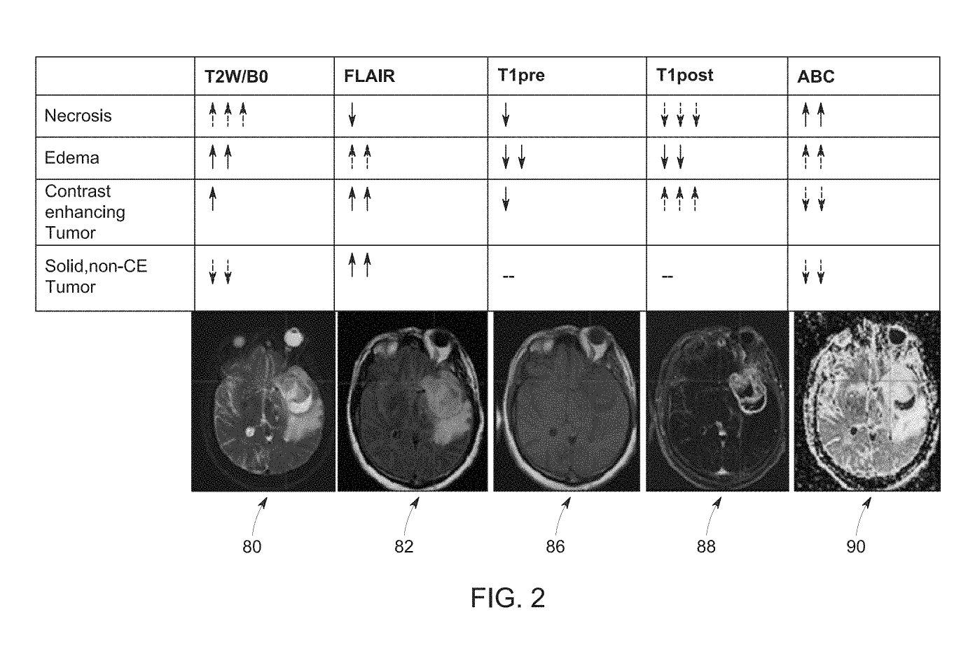

As noted above, to facilitate explanation, an example is described herein related to the use of multi-parametric data from a magnetic resonance imaging (MRI) oncology protocol. An oncology protocol may consist of using images including, but not limited to: T2 weighted (T2W) images which favor imaging water content within structures and are used to demonstrate pathology, T1 weighted (T1W) images in which fat and hydrogen containing structures appear bright or with high-intensity, diffusion weighted images (DWI) and apparent diffusion coefficient (ADC) images which convey intensity proportional to the diffusion constant of water, T1W post-contrast images which conveys T1W image information after administration of a contrast agent, and fluid attenuated inversion recovery (FLAIR) images in which the signal from water is reduced by timing the delay of an inversion pulse. Each of these protocols may convey redundant or complementary information, as illustrated in FIG. 2. In particular, FIG. 2 shows, along the bottom row, images acquired using T2W/B0 (T2 image 80), FLAIR (FLAIR image 82, T1 Pre-contrast (T1 image 86), T1 post-contrast (T1 Post image 88), and ADC (ADC image 90) protocols. As may be appreciated by reviewing these sample images, the differing protocols contain complementary as well as redundant information with respect to the tumor as well as with respect to other structural and/or functional information.

With this example in mind, the present approach removes the redundancy in such multi-parametric image data and utilizes the complementarity and labels the physiological components using known correspondences between the image data (MRI data in this example) and physiological features or properties. Examples of such physiological features or properties include, but are not limited to: e.g., edema, necrosis or necrotic tissue, inflamed tissue, infarcted tissue, cellularity, and so forth. By way of example, the attached table above the images shown in FIG. 2 relates examples of such physiological or biological interpretations and the contribution of the related image types to assessing such physiological or biological interpretations, with the listed examples including necrosis, edema, contrast enhancing tumor, and solid, non-contrast enhancing tumor. Per the table, the strength or contribution provided by the image type for a given interpretation is indicated by the number of up or down arrows (one to three arrows), while dashed arrows and solid arrows, respectively indicate whether the corresponding image intensity is indicated by respective high (i.e., bright) or low (i.e., dark) intensity within the corresponding images. For example, T1 post-contrast image 88 positively indicates contrast enhanced regions (e.g., a contrast enhancing tumor) both strongly (three up arrows) and in high intensity (i.e., as bright regions).

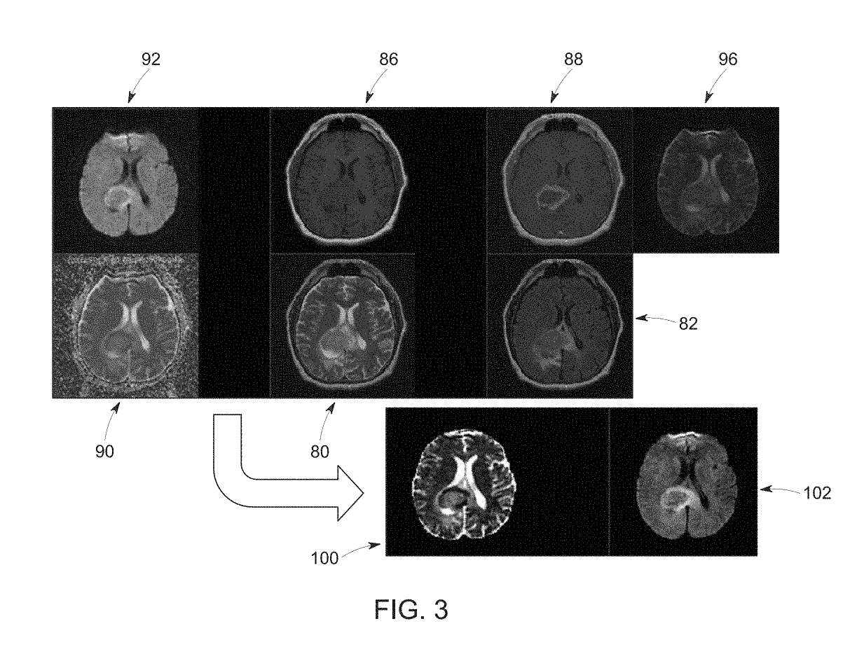

The present approach may employ steps as discussed herein. For example, in one implementation, data reduction methods or data transformation methods (e.g., principal component analysis (PCA), independent component analysis (ICA), non-negative matrix factorization (NNMF), convex analysis of mixtures with non-negative sources (CAMNS), and so forth) that may be implemented in terms of whole data or specific regions of interest (ROI), such as a brain mask or tumor mask, are employed. By way of a present example, NNMF was applied over entire brain image data to initially obtain the decomposition of images into known physiological components (e.g., edema, cellularity and so forth). The NNMF provided the weight matrices as well as the basis source vectors. In this example, illustrated in FIG. 3, seven multi-parametric MRI data images (here DWI image 92, T1 image 86, T1-post image 88, DWI-B0 image 96, ADC image 90, T2 image 80, and FLAIR image 82) are used to generate two physiological component or feature images, i.e., physiology labeled images. The first example is an edema image 100 corresponding to edema in the imaged region. The second example is a cellularity image 102. In this example, the edema image 100 is generated from the weighted multi-parametric images in accordance with: Edema=T2-bright, DWI-B0-bright, T1Pre-dark regions. Similarly, the tumor cellularity image 102 is generated from the multi-parametric images in accordance with: Cellularity=ADC−darkened, Contrast enhancing, T2-dark image regions. The physiology labeled images (e.g., images 100, 102) may be presented to a reviewer (e.g., a clinician or biologist) to characterize the oncology data and/or derive biological correlation out of it.

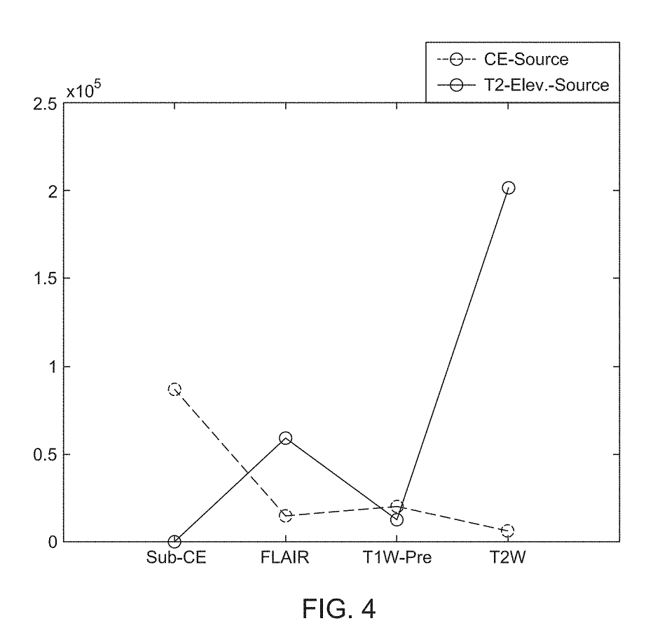

The component or compressed data may then be labeled accordingly as belonging to respective physiological components or features. The labeling can utilize data learned from the imaging data itself (e.g., signal, texture of reduced spatial components) or learned from the source vectors from the data reduction methods. By way of example, in one implementation, labelling of these images can be performed by analysis of the basis vector images as shown in FIG. 4. Each basis vector corresponds to the signature of the physiological component associated with it. Thus, the contrast enhanced (CE) source vector plot, which has elevated values for contrast enhancing data (T1-post or Sub-CE) and low values for T2W data is readily identified as cellularity image data while the T2-elevated component, which has elevated values for FLAIR and T2W is identified as edema image components. This feature vector analysis can be further automated using machine and deep learning based algorithms. Thus, in an alternative approach, a deep learning network is trained to work on the physiology source images to label them as belonging to a physiology component class.

With the preceding in mind, the present approach employs a generic methodology for transforming individual modality specific multi-parametric data into data, e.g., maps or images, which provides direct insight into the underlying physiology of the tissue. This may facilitate better clinical evaluation of the disease data as well as help non-imaging technologists and scientist to directly correlate imaging findings with basic biological phenomenon being studied with imaging. For example, untrained reviewers, may be confused by numerous contrast mechanisms of imaging data, their values, and their interpretation when studying biological processes (e.g., proliferation in tumors or gene expression involved in inflammation). So, presenting the imaging data in a format which can be directly correlated to biology (e.g., necrosis, edema, and so forth) will accelerate research activities using different radiological imaging modalities and wider acceptance in the community.

Technical effects of the invention include transforming multi-parametric image data into images explicitly conveying physiology or physiological function, such as edema, cellularity, or tissue necrosis images or maps. Such images address problems related to untrained reviewers being able to ascertain and interpret biologically or physiologically relevant information from such image data. In this manner, multi-modality radiology data may be presented in terms of standardized information sought by clinicians and biologists i.e., the under-lying patho-physiology of the tissue

This written description uses examples to disclose the invention, including the best mode, and also to enable any person skilled in the art to practice the invention, including making and using any devices or systems and performing any incorporated methods. The patentable scope of the invention is defined by the claims, and may include other examples that occur to those skilled in the art. Such other examples are intended to be within the scope of the claims if they have structural elements that do not differ from the literal language of the claims, or if they include equivalent structural elements with insubstantial differences from the literal languages of the claims.

1. A method for generating a physiology labeled image, comprising:
acquiring two or more multi-parametric images of a subject, wherein the two or more multi-parametric images are acquired using different imaging protocols;
performing a data reduction analysis on the two or more multi-parametric images, wherein the outputs of the data reduction analysis comprises computational products of the two or more images into one or more physiological components;
generating the physiology labeled image based on the computational products; and
displaying the physiology labeled image for review.

2. The method of claim 1, wherein the multi-parametric images are acquired using one of a magnetic resonance imaging (MRI) system; a computed tomography (CT) imaging system, an ultrasound imaging system, a positron emission tomography (PET) imaging system, or a single photon emission computed tomography (SPECT) imaging system.

3. The method of claim 1 wherein the multi-parametric images are acquired using a magnetic resonance imaging (MRI) system and comprise one or more of T2 weighted (T2W) images, T1 weighted (T1W) images, diffusion weighted images (DWI), apparent diffusion coefficient (ADC) images, T1W post-contrast images, and fluid attenuated inversion recovery (FLAIR) images.

4. The method of claim 1, wherein the two or more multi-parametric images contain redundant or complementary information with respect to a physiological structure or function of interest.

5. The method of claim 1, wherein the physiology labeled image comprises one or more of an edema image, a necrosis image, an inflamed tissue image, an infarcted tissue image, or a cellularity image.

6. The method of claim 1, wherein the data reduction analysis comprises one or more of principal component analysis (PCA), independent component analysis (ICA), non-negative matrix factorization (NNMF), or convex analysis of mixtures with non-negative sources (CAMNS).

7. The method of claim 1, wherein the computational products comprise one or both of weight matrices and basis source vectors for the one or more physiological components.

8. The method of claim 1, wherein the computational products correspond to a signature of the one or more physiological components.

9. An image processing system, comprising:
a processor configured to execute executable instructions; and
a memory configured to store executable instructions that, when executed by the processor, cause act to be performed comprising:
acquiring or accessing two or more multi-parametric images of a subject, wherein the two or more multi-parametric images are acquired using different imaging protocols;
performing a data reduction analysis on the two or more multi-parametric images, wherein the outputs of the data reduction analysis comprises computational products of the two or more images into one or more physiological components;
generating a physiology labeled image based on the computational products; and
displaying the physiology labeled image for review.

10. The image processing system of claim 9, wherein the multi-parametric images are acquired using one of a magnetic resonance imaging (MRI) system; a computed tomography (CT) imaging system, an ultrasound imaging system, a positron emission tomography (PET) imaging system, or a single photon emission computed tomography (SPECT) imaging system.

11. The image processing system of claim 9, wherein the two or more multi-parametric images contain redundant or complementary information with respect to a physiological structure or function of interest.

12. The image processing system of claim 9, wherein the physiology labeled image comprises one or more of an edema image, a necrosis image, an inflamed tissue image, an infarcted tissue image, or a cellularity image.

13. The image processing system of claim 9, wherein the data reduction analysis comprises one or more of principal component analysis (PCA), independent component analysis (ICA), non-negative matrix factorization (NNMF), or convex analysis of mixtures with non-negative sources (CAMNS).

14. The image processing system of claim 9, wherein the computational products comprise one or both of weight matrices and basis source vectors for the one or more physiological components.

15. The image processing system of claim 9, wherein the computational products correspond to a signature of the one or more physiological components.

16. One or more non-transitory computer readable media encoding routines which, when executed, cause acts to be performed comprising:
acquiring two or more multi-parametric images of a subject, wherein the two or more multi-parametric images are acquired using different imaging protocols;
performing a data reduction analysis on the two or more multi-parametric images, wherein the outputs of the data reduction analysis comprises computational products of the two or more images into one or more physiological components;
generating a physiology labeled image based on the computational products; and
displaying the physiology labeled image for review.

17. The one or more non-transitory computer readable media of claim 16, wherein the physiology labeled image comprises one or more of an edema image, a necrosis image, an inflamed tissue image, an infarcted tissue image, or a cellularity image.

18. The one or more non-transitory computer readable media of claim 16, wherein the data reduction analysis comprises one or more of principal component analysis (PCA), independent component analysis (ICA), non-negative matrix factorization (NNMF), or convex analysis of mixtures with non-negative sources (CAMNS).

19. The one or more non-transitory computer readable media of claim 16, wherein the computational products comprise one or both of weight matrices and basis source vectors for the one or more physiological components.

20. The one or more non-transitory computer readable media of claim 16, wherein the computational products correspond to a signature of the one or more physiological components.
